(19) United States
(12) Patent Application Publication
(10) Pub. No.: US 2018/0102188 A1
(43) Pub. Date: Apr. 12, 2018

(54) METHOD FOR ASSISTING PATIENTS IN NAVIGATING A HEALTHCARE NETWORK FROM PRE-PROCEDURE THROUGH POST-ADMISSION

(71) Applicant: Innovative Healthcare Delivery LLC, Las Vegas, NV (US)

(72) Inventor: Shelli Lara, Las Vegas, NV (US)

(21) Appl. No.: 15/600,869

(22) Filed: May 22, 2017

Related U.S. Application Data
(63) Continuation of application No. 14/212,129, filed on Mar. 14, 2014, now abandoned.
(60) Provisional application No. 61/792,975, filed on Mar. 15, 2013.

(51) Int. Cl. G06Q 50/24

(52) U.S. Cl. CPC G16H 15/00; G16H 10/60; G16H 20/10

(57) ABSTRACT

In an embodiment, a first communication that is an electronic hospital census and/or an outpatient authorization report is received by a control device operated by an entity that is a different entity than the hospital. The first communication identifies an individual admitted to a hospital and/or individual scheduled for an elective outpatient procedure. The control device stores a file for the patient identifying a service needed by the patient, and the service is a pre-procedure service, a post-procedure service, a pre-discharge service, and/or a post-discharge service. The entity that operates the control device performs a second communication that coordinates the service from a provider that is a different entity than the entity that operates the control device. The second communication is performed at a time that is at least one of before admission to the hospital, at admission to the hospital, before the procedure, after the procedure, or at discharge.

### PRIORITY CLAIM

This application is a continuation of U.S. patent application Ser. No. 14/212,129 filed on Mar. 14, 2014, which claims priority to U.S. Provisional Patent Application Ser. No. 61/792,975 filed on Mar. 15, 2013, the entire contents of which is incorporated herein by reference and relied upon.

### BACKGROUND

Medical care is necessary to investigate and/or treat pathological conditions such as a disease and/or an injury or to help improve bodily function. However, the complex infrastructure of the healthcare delivery system can create barriers that often arise during the transition from the hospital to the outpatient setting and from planned outpatient procedures. Health literacy impediments, poor medication adherence, failures to visit the primary care physician or appropriate specialist, and accessing necessary care or services in a timely manner can increase the risk of avoidable readmissions and delays in patient recovery. Ultimately, these barriers create inefficiencies in the healthcare system that result in increased costs associated with poor quality care.

### SUMMARY

In a general embodiment, a method is provided. The method comprises: receiving a first communication selected from the group consisting of an electronic hospital census and an outpatient authorization report, the first communication identifying a patient selected from the group consisting of an individual admitted to a hospital, an individual scheduled for an elective outpatient procedure, and combinations thereof, the first communication received by a control device operated by an entity that is a different entity than the hospital; determining a service needed by the patient identified by the first communication, the service selected from the group consisting of a pre-procedure service, a post-procedure service, a pre-discharge service, a post-discharge service and combinations thereof, the control device storing a file for the patient that identifies the post-procedure service; and performing a second communication that coordinates the service from a provider that is a different entity than the entity that operates the control device, the second communication performed by the entity that operates the control device, and the second communication is performed at a time selected from the group consisting of before admission to the hospital, at admission to the hospital, before the procedure, after the procedure, at discharge, and combinations thereof.

In an embodiment, the method further comprises displaying information related to the service on a web page provided by the entity and accessible to the patient.

In an embodiment, the method further comprises generating a census alert, and the control device generates the census alert in response to processing of the first communication.

In an embodiment, the service is related to prescriptions, and the second communication is conducted with a pharmacy and/or with an entity managing or controlling the pharmacy benefit plan.

In an embodiment, the service is a medical appointment. In a related embodiment, the second communication schedules the medical appointment, and the patient receives an alert on the day of or prior to the medical appointment, the alert identifying information regarding the medical appointment and sent by the entity that operates the control device.

In an embodiment, the service is an appointment with a vendor.

In an embodiment, the method further comprises automatically generating a discharge alert when the patient is discharged, and the control device automatically generates the discharge alert in response to the first communication identifying that the patient is discharged.

In an embodiment, the control device is communicatively connected to a navigation specialist, and the navigation specialist performs the second communication that coordinates the service.

In an embodiment, the method further comprises updating the file, and the control device updates the file in response to information input on a navigation specialist remotely located relative to the control device.

In an embodiment, the method further comprises identifying a plurality of services comprising the service and an associated completion status for each of the plurality of services on a web page accessible by the patient, the web page provided by the entity that operates the control device.

In an embodiment, the method further comprises providing information from the file on a web page accessible to the patient.

In an embodiment, the method further comprises generating a call alert that requests contact between the patient and a navigation specialist, the entity that operates the control device comprises the navigation specialist, and the control device generates the call alert.

In an embodiment, the method further comprises obtaining a medical record for the patient, the control device electronically transmitting the medical record to a third party associated with a pre-procedure service.

In an embodiment, the method further comprises obtaining a medical record for the patient, the control device electronically transmitting the medical record to a third party associated with a post-procedure service.

In another embodiment, a system is provided. The system comprises a control device configured to receive a first communication selected from the group consisting of an electronic hospital census and an outpatient authorization report, the first communication identifying a patient selected from the group consisting of an individual admitted to a hospital, an individual scheduled for an outpatient procedure (e.g., an elective or a non-elective outpatient procedure), and combinations thereof, and the control determines a service needed by the patient identified by the first communication, the service selected from the group consisting of a pre-procedure service, a post-procedure service, a pre-discharge service, a post-discharge service and combinations thereof, the control device storing a file for the patient that identifies the service, the control device operated by an entity that is a different entity than the hospital. The system further comprises a navigation specialist communicatively connected to the control device and operated by the entity that operates the control device, and at least one of the control device and the navigation specialist performs a second communication that coordinates the service from a provider that is a different entity than the entity that operates the control device and the navigation specialist, and the second communication is performed at a time selected from the group consisting of before admission to the hospital, at admission to the hospital, before the procedure, after the procedure, at discharge, and combinations thereof.

In an embodiment, the navigation specialist is remotely located relative to the control device.

In another embodiment, a non-transitory computer-readable medium is provided. The computer-readable medium stores instructions that when executed cause a processor to perform at least one step of the method.

In an embodiment, the control device is remotely located relative to the hospital.

In an embodiment, the method further comprises obtaining a medical record resulting from the service coordinated by the second communication, the control device electronically transmitting the medical record to a third party associated with another service needed by the patient.

In an embodiment, the entity that operates the control device performs the second communication before admission of the patient to the hospital.

In an embodiment, the entity that operates the control device performs the second communication before the procedure.

In an embodiment, the entity that operates the control device performs the second communication before the procedure.

In an embodiment, the entity that operates the control device performs the second communication before the patient is discharged.

One or more embodiments advantageously reduce readmission to the hospital after a hospital admission.

In one embodiment, the method and system improve efficiencies and/or cost outcomes in a healthcare system.

In one embodiments, the method and system align appropriate resources to facilitate a value-based healthcare delivery system.

In another embodiment, the method and system ensure pre- and post-procedure care coordination for patients receiving an outpatient procedure (e.g., an elective or a non-elective outpatient procedure).

In other embodiments, the method and system provide real-time support to patients and their families.

In other embodiments, the method and system provide a comprehensive and proactive patient-centered approach that begins before the procedure and continues through post-procedure treatment.

In other embodiments, the method and system provide early intervention and care coordination initiated before a procedure.

In other embodiments, the method and system reduce disruptions in post-operative care, identify barriers to healthcare, and provide support to patients in an outpatient setting.

In other embodiments, the method and system allow patients to focus on recovery, educate patients on the recovery process, financial impact and overall treatment plan, and provide clear expectation regarding same.

In other embodiments, the method and system proactively identify and address post-procedure complications and promote collaboration by physicians, such as physicians of different disciplines.

In other embodiments, the method and system obtain continuity of healthcare by involving healthcare providers that will be interacting with the patient and providing information to same.

In another embodiment, the method and system increases a probability or ensures that a patient visits a primary care physician after the procedure.

In another embodiment, the method and system ensures that prescriptions, orders for home health and follow-up appointments are scheduled prior to the procedure and, after the procedure, and confirms that the services have been provided in a timely manner.

### BRIEF DESCRIPTION OF THE FIGURES

FIG. 1 depicts an example inpatient process in a transition care process according to an embodiment of the present invention.

FIGS. 2A-2B depicts an example discharge process in a transition care process according to an embodiment of the present invention.

FIGS. 3A-3B depicts an example care assist process according to an embodiment of the present invention.

### DETAILED DESCRIPTION

As used in this specification, the singular form of a word includes the plural, and vice versa, unless the context clearly dictates otherwise. Thus, the references “a”, “an”, and “the” are generally inclusive of the plurals of the respective terms. For example, reference to “an embodiment” or “a method” includes a plurality of such “embodiments” or “methods.” Similarly, the words “comprise”, “comprises”, and “comprising” are to be interpreted inclusively rather than exclusively. Likewise the terms “include”, “including” and “or” should all be construed to be inclusive, unless such a construction is clearly prohibited from the context. The terms “comprising” or “including” are intended to include embodiments encompassed by the terms “consisting essentially of” and “consisting of. ” Similarly, the term “consisting essentially of” is intended to include embodiments encompassed by the term “consisting of”.

As used herein, the term “procedure” means any medical procedure provided to the patient in a hospital, provider's office, outpatient facility, long term facility, or the like, including propedeutic procedures such as observation of the patient. Thus, although examples of procedures include surgical procedures, therapeutic procedures, and diagnostic procedures, the present invention is not limited to these examples of procedures.

As used herein, the term “navigation specialist” refers to an individual or an individual assisted by a machine (e.g., a terminal connected to a system as described herein) who interacts with one or more of: a patient, a physician, a clinician, a hospital, a medical appointment provider, a transportation provider, a pharmacy, or any other suitable entity or individual on behalf of a patient. In some embodiments, the navigation specialist is employed by a third party other than a hospital. In some embodiments, the navigation specialist is employed by a medical appointment provider. In some embodiments, the navigation specialist is employed by a hospital.

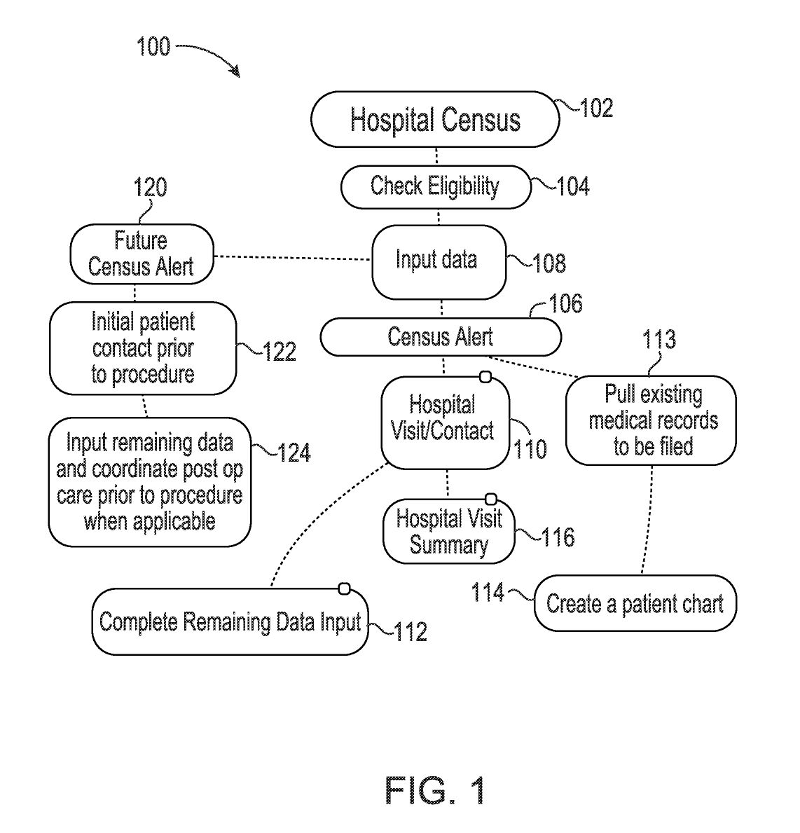

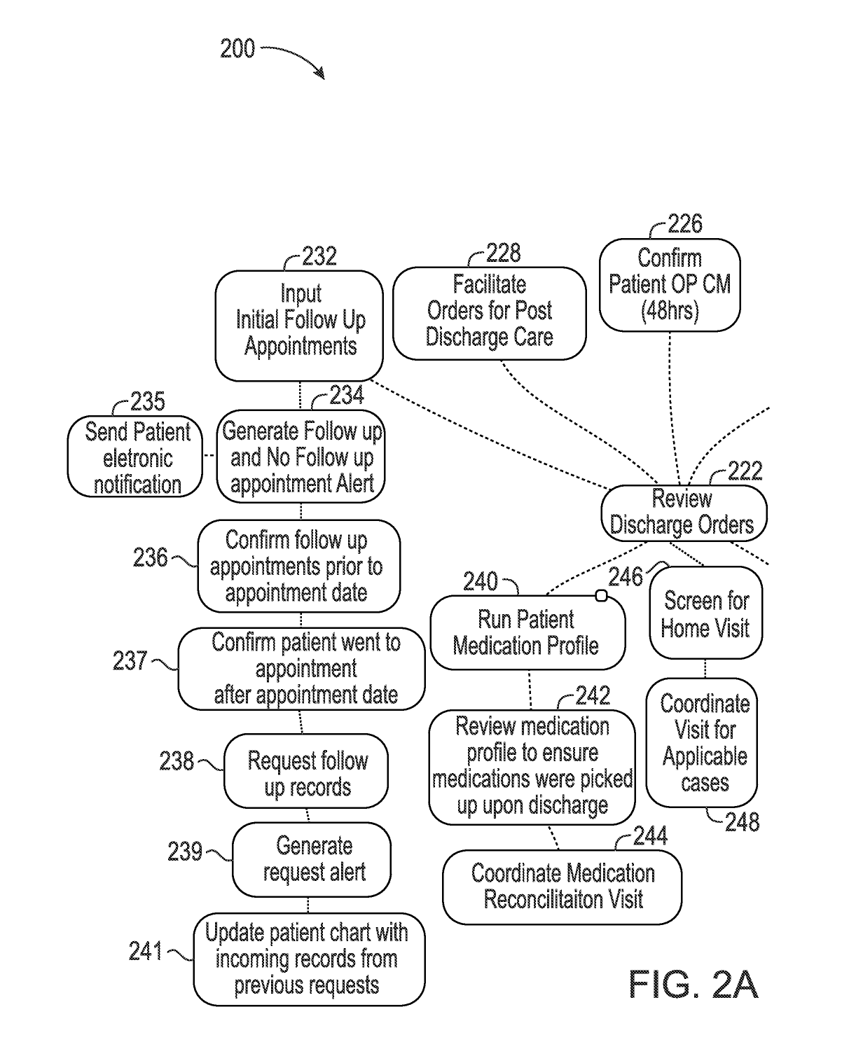

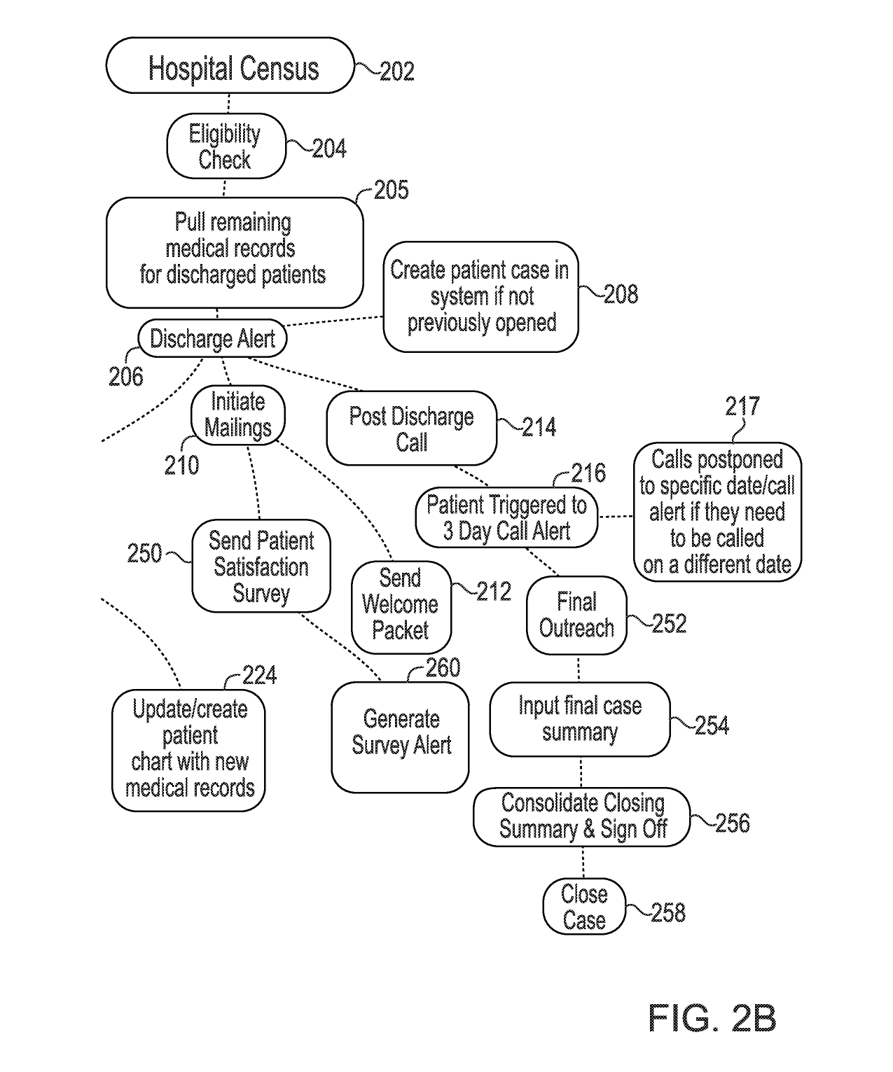

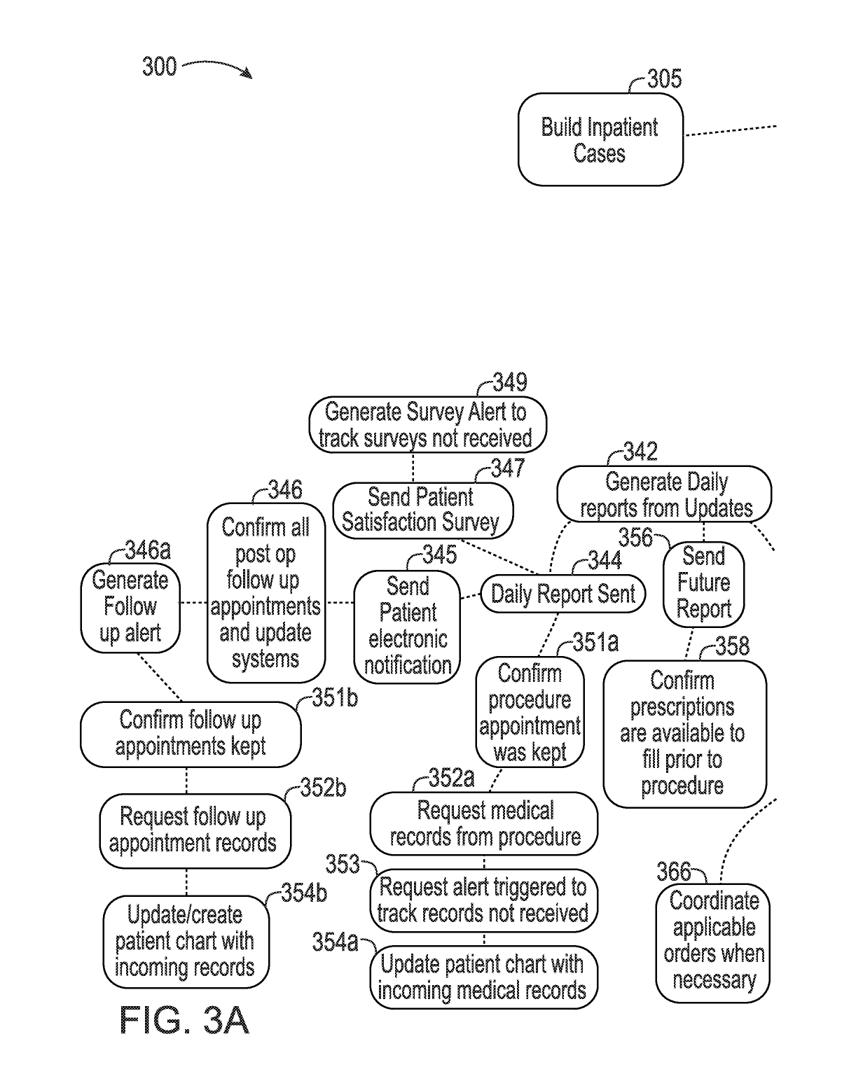

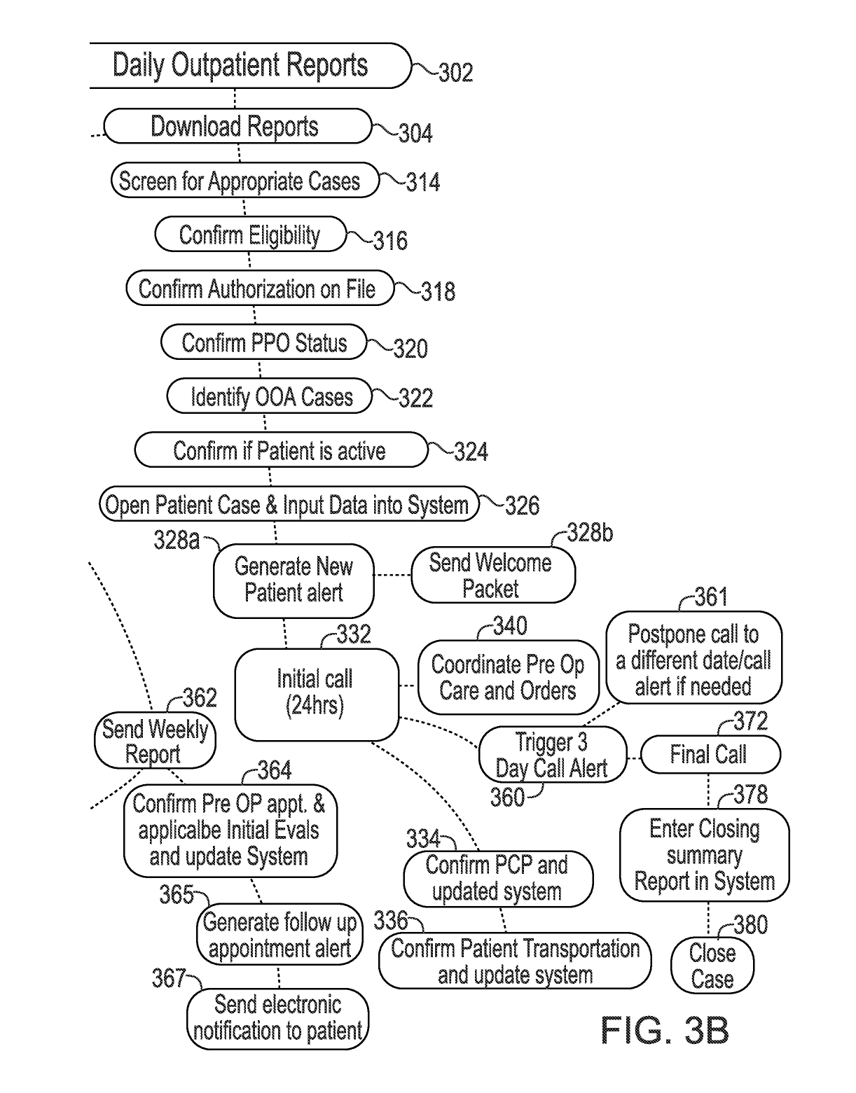

Referring now to FIGS. 1 to 3B, a patient may participate in a transition care method 100/200 (FIGS. 1 to 2B, respectively) and/or a care assist program 300 (FIGS. 3A to 3B) by registering with the entity that operates a method consistent with the present disclosure, or an entity that controls a system consistent with the present disclosure. In one embodiment, the patient registers by executing an agreement describing the transition care method 100/200 and/or the care assist program 300 and setting forth terms and conditions. The registration of the patient enables data associated with patient to be provided, obtained, shared, and/or processed before the patient participates in a transition care method and/or a care assist program consistent with the present disclosure. Such data may include and/or may identify a procedure to be received by the patient and/or the type of health insurance owned by the patient, if any.

The transition care method 100/200 may be used for patients that are hospitalized for at least one overnight stay (“inpatient treatment”), and the transition care method 100/200 may comprise an inpatient process 100 and/or a discharge process 200. FIG. 1 is a flowchart of an embodiment of the inpatient process 100. Although the inpatient process 100 is described with reference to the flowchart illustrated in FIG. 1, other methods of performing the inpatient process 100 may be used. For example, unless explicitly stated otherwise, the order of the steps may be changed, and each of the steps is independently optional.

In some embodiments, a hospital census 102 is obtained or received. In some embodiments, the hospital census 102 includes information displayed in a predetermined format. In some embodiments, the hospital census 102 comprises a list of patients and optionally further comprises data associated with each of the listed patients. In some embodiments, the data comprises one or more of a patient name, a diagnosis, an admission date, an attending doctor, a brief summary of EMR notes, if the patient has been or is in case management, and the like. In some embodiments, the list of patients in the hospital census 102 comprises one or more of newly admitted patients, patients previously admitted but not yet discharged, or newly discharged patients. In embodiments in which the hospital census 102 is associated with a specific facility, the hospital census 102 may identify all patients admitted the day the hospital census is generated and/or all patients from the previous hospital census 102 that are still admitted. In an embodiment, the list of patients in the hospital census 102 comprises patients that previously registered to participate in the transition care method 100/200. In some embodiments, the list of patients in the hospital census 102 comprises patients for whom the procedure at the hospital have already been identified. In some embodiments, the eligibility of the listed patients for the subsequent steps of the inpatient process 100 is confirmed. In some embodiments, eligibility is based at least in part on the insurance coverage of the patient, the procedure to be received by the patient, and/or the like.

In an embodiment, the hospital census 102 is obtained or received from a hospital or from a plurality of hospitals, for example, in electronic format. In some embodiments, the hospital census 102 is obtained or received in a fax, an email, a secure email, an attachment to an email, a text message, an instant message, an FTP communication, a webpage for which an address and/or a link is transmitted, a remote portal, a text message, an automated telephone call, a live telephone call, a voicemail, a video communication, a communication over a network, and/or a written communication.

In some embodiments, the hospital census 102 is obtained or received at a predetermined frequency, a predetermined day and/or a predetermined time. In some embodiments, the hospital census 102 is obtained or received daily, such as daily at 5:00 AM. In another embodiment, the hospital census 102 is obtained or received within a predetermined frequency threshold and/or a predetermined time threshold. In some embodiments, the hospital census 102 is received or obtained no later than 8:30 AM daily.

In some embodiments, the hospital census 102 is processed automatically. In an embodiment, the hospital census 102 is processed at a predetermined frequency, a predetermined day and/or a predetermined time. In some embodiments, the hospital census 102 is processed daily, such as daily at about 8:30 AM. In another embodiment, the hospital census 102 is processed within a predetermined frequency threshold and/or a predetermined time threshold. In some embodiments, the hospital census 102 is processed by no later than about 8:30 AM daily. In an embodiment, processing of the hospital census begins (e.g., automatically begins) upon receipt of the hospital 102.

In some embodiments, as shown in Step 104, the hospital census 102 is processed to confirm eligibility of the patients identified in the hospital census 102. In some embodiments, the eligibility is based at least in part on the insurance coverage of the patient and/or one or more procedures to be received by the patient. In an embodiment, eligibility is based at least partially on the patient being eligible under their health plan. In some embodiments, a patient identification process that identifies the patient eligible to participate in subsequent steps of the inpatient process 100 comprises one or more of Steps 102 and 104.

In some embodiments, as shown in Step 108, data from Steps 102 and/or 104 is input into a file (e.g., an electronic data file) which is stored in a system consistent with the present disclosure. In some embodiments, the file includes information specific to a single patient (e.g., one newly admitted patient) identified in the census alert 106. In some embodiments, a file is generated and/or stored in a system of the present disclosure for each of the newly admitted eligible patients identified in Step 102 and/or Step 104. In an embodiment, the file is generated (e.g., automatically generated) in response to the census alert 106 and/or the completion of the eligibility check in Step 104. In some embodiments, the file comprises data from the hospital census 102, and may optionally be at least partially populated with the data obtained when the patient registered for participation in the transition care method 100/200. In some embodiments, at least a portion of the file is electronically displayed to the patient and/or the navigation specialist, for example on a web page, mobile device, cellular device, computer, tablet, or any other suitable electronic display screen. In some embodiments, the file is at least partially displayed to the patient on a web page.

In one embodiment, the file is generated in Step 108 for the newly admitted patient(s) within a predetermined time threshold relative to receipt of the hospital census 102 identifying the patient as newly admitted and eligible (e.g., in Step 104). In some embodiments, the file is generated for a patient on the same day that the hospital census 102 identifying the patient as newly admitted and eligible is received or obtained. In some embodiments, at least a portion of the file is generated before the patient is admitted, such as when the patient registers for participation in the transition care method 100/200. In some embodiments, the file may be generated before the procedure.

In some embodiments, a census alert 106 is generated. In some embodiments, the census alert 106 identifies one or more patients as eligible for subsequent steps of the inpatient process 100. In some embodiments, the census alert 106 identifies one or more patients as newly admitted. In some embodiments, the census alert 106 identifies one or more patients previously admitted to the hospital and not yet discharged from the hospital. In one embodiment, each patient identified as eligible in Step 104 is identified in a census alert 106. In some embodiments, the census alert 106 identifies the name of the patient, the diagnosis of the patient, the physician for the patient, whether the patient is in case management, and/or the like. In some embodiments, the census alert 106 is automatically generated upon processing of the hospital census 102 in Step 104.

In some embodiments, the census alert 106 is automatically transmitted in response to the data input of Step 108. In some embodiments, the census alert 106 is transmitted to a navigation specialist and/or stored in the system. In some embodiments, the census alert 106 generated in response to the data input of Step 108 are automatically provided in response to the data input of Step 108. In some embodiments, the census alert 106 is transmitted to the navigation specialist and/or stored on the system in a fax, an email, a secure email, an attachment to an email, a text message, an instant message, an FTP communication, a webpage for which an address and/or a link is transmitted, a remote portal, a text message, an automated telephone call, a live telephone call, a voicemail, a video communication, a communication over a network, and/or a written communication.

In an embodiment, the census alert 106 is generated at a predetermined frequency, a predetermined day and/or a predetermined time. In some embodiments, the census alert 106 is generated daily, such as daily at 8:30 AM. In another embodiment, the census alert 106 is generated within a predetermined frequency threshold and/or within a predetermined time threshold. In some embodiments, the census alert 106 is generated no later than 8:30 AM daily. In one embodiment, the census alert 106 is generated in response to receipt of the hospital census 102 and/or completion of processing of the hospital census in Step 104.

In some embodiments, the navigation specialist may facilitate an introductory patient visit 110 at the hospital. In some embodiments, the introductory patient visit 110 is in person and face-to-face. In some embodiments, the introductory patient visit 110 is performed within a predetermined time threshold, such as, for example, the day that the patient is admitted to the hospital and/or the same day as the census alert 106. In some embodiments, the introductory patient visit 110 is conducted in Step 110 before the procedure and/or before the patient has been discharged. In some embodiments, the introductory patient visit 110 is prompted and/or performed based on information included in the census alert 106.

In some embodiments, the introductory patient visit 110 comprises confirming if the newly admitted patient feels comfortable discussing admission with others present in the room; providing contact information associated with the navigation specialist; explaining the inpatient process 100 and/or the discharge process 200; providing materials explaining the inpatient process 100 and/or the discharge process 200; confirming contact information for the newly admitted patient, such as a telephone number; confirming that the newly admitted patient has a primary care physician; confirming that the newly admitted patient understands their discharge needs; identifying the most recent time the patient was seen by the admitting physician and/or the specialist; and/or determining if the current hospital admission is related to a chronic problem, an acute problem or a new diagnosis. If the patient does not have a primary care physician, introductory patient visit 110 may further comprise coordinating a primary care physician for the patient. In some embodiments, the introductory patient visit 110 is conducted within a predetermined time threshold, for example on the same day as the census alert 106 for the newly admitted patient is generated. In one embodiment, the introductory patient visit 110 is conducted (e.g., automatically coordinated and subsequently conducted) upon generation of the census alert 106.

The navigation specialist may enter the information from the introductory patient visit 110 into a system as described herein. In some embodiments, at least a portion of the information from the introductory patient visit 110 is electronically displayed to the navigation specialist and/or to the patient, for example on a web page accessible by the navigation specialist and/or the patient. In some embodiments, the system automatically organizes the information to be electronically displayed. In some embodiments, the information is organized (e.g., automatically organized) differently depending on whether the information will be electronically displayed to the navigation specialist or to the patient.

In some embodiments, such as shown in Step 112, additional data is optionally obtained after the introductory patient visit. In some embodiments, the additional data is obtained from a third party, by consulting hospital personnel, and/or any from any other source of additional information related to or associated with the patient. In some embodiments, the additional information comprises data not yet contained in the file stored by the system. In some embodiments, the additional data comprises the identity of a primary care physician, the identity of a specialist, the identity of an ancillary provider, and/or demographics associated with the patient. In an embodiment, the navigation specialist may obtain at least a portion of the additional data. In some embodiments, system may provide instructions related to obtaining the additional data to the navigation specialist. For example, the instructions may be transmitted to the navigation specialist in the census alert 106.

The navigation specialist may enter the additional data obtained in Step 112 into the system. The additional data may be transmitted to, obtained by and/or stored by the system. In some embodiments, the system organizes the additional data and/or electronically displays at least a portion of the additional data to the navigation specialist and/or to the patient, for example on a web page. In some embodiments, the system automatically organizes the additional data. In some embodiments, the additional information is organized (e.g., automatically organized) differently depending on whether the information will be electronically displayed to the navigation specialist or to the patient. In some embodiments, the additional data is incorporated into the file for the patient stored by the system.

In some embodiments, the information from the introductory patient visit 110 and/or the additional data may be transmitted in a fax, an email, a secure email, an attachment to an email, a text message, an instant message, an FTP communication, a webpage for which an address and/or a link is transmitted, a remote portal, a text message, an automated telephone call, a live telephone call, a voicemail, a video communication, a communication over a network, and/or a written communication.

In some embodiments, as shown in Step 113, preexisting medical records are obtained, located or received in response to census alert 106. In Step 114, a patient chart is created for one or more patients identified in census alert 106, optionally including the preexisting medical records obtained, located or received in Step 113. In some embodiments, the patient chart identifies status changes of the patient, measurements of parameters of the patient, and the like, and may be updated periodically. In an embodiment, the patient chart is created at least partially by the navigation specialist and/or the system. In a related embodiment, instructions related to creating the patient chart are provided to the navigation specialist and/or the system. In some embodiments, the instructions are transmitted to the navigation specialist and/or the system in census alert 106 and/or in a separate message generated in response to census alert 106.

In some embodiments, information from the patient chart, including updates to the patient chart, is transmitted to, obtained by and/or stored by the system. In some embodiments, the information from the patient chart is electronically displayed to the patient and/or the navigation specialist, for example on a web site. In some embodiments, the patient chart is incorporated into the file for the patient stored by the system.

In some embodiments, information in the patient chart and/or any other patient information is used to follow (e.g., monitor) progress of the patient and/or to determine if another hospital visit is needed. In some embodiments, the patient chart and discussions with the charge nurse and/or a hospital case management individual are used to follow the progress of the patient and/or to determine if another hospital visit is needed. In some embodiments, determining if another hospital visit is needed is based at least in part on a response to the census alert 106. In some embodiments, census alert 106 is received or obtained daily, and determining whether another hospital visit is needed is also assessed daily.

In some embodiments, a hospital visit summary 116 is created In some embodiments, the hospital visit summary 116 is created within a predetermined time threshold, such as within twenty-four hours of when the patient is discharged. In some embodiments, at least a portion of the hospital visit summary 116 is created automatically, for example upon receipt of one or more alerts or reports. In some embodiments, the hospital visit summary 116 comprises a hospital discharge summary, associated physician information, demographics and/or information related to clinicals. In an embodiment, at least a portion of the hospital visit summary is created by the navigation specialist and/or the system. In some embodiments, instructions for creating the hospital visit summary 116 are provided to the navigation specialist and/or to the system, for example in census alert 106.

In some embodiments, the hospital visit summary 116 is transmitted to, obtained by and/or stored by the system. In some embodiments, the hospital visit summary is organized and/or electronically displayed to the navigation specialist and/or the patient. In some embodiments, the hospital visit summary 116 is incorporated into the file for the patient stored by the system. In some embodiments, the hospital visit summary 116 is transmitted in a fax, an email, a secure email, an attachment to an email, a text message, an instant message, an FTP communication, a webpage for which an address and/or a link is transmitted, a remote portal, a text message, an automated telephone call, a live telephone call, a voicemail, a video communication, a communication over a network, and/or a written communication.

In some embodiments, a process for service facilitation during a hospital stay comprises at least one of Step 110, Step 112, Step 114, Step 116 or a combination thereof. In some embodiments, at least one of Step 110, Step 112, Step 114 and Step 116 is/are performed during the time period from patient admittance to patient discharge. In one embodiment, Step 110, Step 112, Step 114 and Step 116 are each performed during the time period from patient admittance to patient discharge.

In some embodiments, a future census alert 120 is generated. In some embodiments, In some embodiments, the future census alert 120 identifies one or more patients that will be admitted to a hospital facility in the future, for example one or more patients that have a scheduled admission or procedure date after the date the future census report is generated. In one embodiment, each patient identified as eligible in Step 104 is identified in census alert 106 or in a future census alert 120. In some embodiments, the future census alert 120 identifies the name of the patient, the diagnosis of the patient, the physician for the patient, whether the patient is in case management, and/or the like. In some embodiments, the future census alert 120 is automatically generated upon processing of the hospital census 102 in Step 104.

In some embodiments, the future census alert 120 is generated and/or transmitted in response to the data input of Step 108. In some embodiments, the future census alert 120 is transmitted to a navigation specialist and/or stored in the system. In some embodiments, the future census alert 120 is automatically provided in response to the data input of Step 108. In some embodiments, the future census alert 120 is transmitted to the navigation specialist and/or stored on the system in a fax, an email, a secure email, an attachment to an email, a text message, an instant message, an FTP communication, a webpage for which an address and/or a link is transmitted, a remote portal, a text message, an automated telephone call, a live telephone call, a voicemail, a video communication, a communication over a network, and/or a written communication.

In some embodiments, as shown in Step 122, a patient identified in a future census alert 120 is contacted prior to a scheduled procedure. In some embodiments, the patient is contacted before admission to the hospital. In other embodiments, the patient is contacted after admission but before the procedure. In some embodiments, the patient is contacted on the same day that future census alert 120 is generated.

In some embodiments, as shown in Step 124, additional information about the patient (e.g., obtained during the patient contact of Step 122), the procedure and/or post-procedure care requirements are input into the file. In some embodiments, the additional information is input into the file before the procedure is performed on the patient.

Any step or any sequence or combination of steps of the inpatient process 100 may be performed automatically upon completion of any other (e.g., any preceding) step. For example and without limitation, the census alert 106 may be generated automatically when the data is input in Step 108. In some embodiments, the present disclosure provides a system configured to automatically perform one or more steps upon completion of any other step of inpatient process 100. In one such embodiment, the system comprises a memory device configured to store instructions which, upon execution, automatically performs one or more steps upon completion of any other step of inpatient process 100. In an embodiment, at least Step 116 is performed automatically by the system; for example, the hospital visit summary 116 is automatically created when the patient is discharged. In an embodiment, information related to the healthcare of the patient and progress of the patient is electronically displayed to the navigation specialist and/or to the patient throughout at least a portion of, or throughout the entirety of, inpatient process 100.

As noted above, the transition care method may comprise the inpatient process 100 and/or the discharge process 200. FIGS. 2A-2B disclose a flowchart of an embodiment of the discharge process 200. Although the discharge process 200 is described with reference to the flowchart illustrated in FIGS. 2A-2B, other methods of performing the discharge process 200 may be used. For example, unless explicitly stated otherwise, the order of the steps may be modified, and each of the steps is independently optional.

One or more of the steps of the discharge process 200 may form a process for service facilitation post-discharge. The discharge process 200 may be performed at least partially after the inpatient process 100, but one or more steps in the discharge process 200 may be performed before, after, or concurrently with one or more steps of the inpatient process 100.

In some embodiments, a hospital census 202 is received or obtained. In some embodiments, the hospital census 202 is received or obtained at a predetermined frequency such as, for example, daily. In some embodiments, the hospital census 202 comprises a fax, an email, a secure email, an attachment to an email, a text message, an instant message, an FTP communication, a webpage for which an address and/or a link is transmitted, a remote portal, a text message, an automated telephone call, a live telephone call, a voicemail, a video communication, a communication over a network, and/or a written communication.

In some embodiments, an eligibility check 204 is performed to confirm the eligibility of the patients identified in the hospital census 202. In some embodiments, at least a portion of the eligibility check 204 is automatically performed, for example upon receipt of the hospital census 202. In some embodiments, the eligibility check 204 comprises determining the eligibility of the patient for subsequent steps of the discharge process 200. In some embodiments, the eligibility check 204 is based at least in part on the insurance coverage of the patient and/or one or more procedures to be received by the patient. In an embodiment, eligibility check 204 is based at least partially on the patient being eligible under their health plan. In some embodiments, a patient identification process that identifies the patient as eligible to participate in subsequent steps of the discharge process 200 comprises one or more of Steps 202 and 204.

In some embodiments, such as shown in Step 205, medical records are obtained or received for patients listed in hospital census 202 and/or patients whose eligibility have been confirmed in eligibility check 204.

In some embodiments, a discharge alert 206 is generated for a patient, such as a patient listed in hospital census 202 and/or patients whose eligibility have been confirmed in eligibility check 204. In an embodiment, each patient identified in the eligibility check 204 is identified in a discharge alert 206. In some embodiments, the discharge alert 206 comprises the patient name, the telephone number, and the facility.

In one embodiment, the discharge alert 206 is provided (e.g., transmitted) to the navigation specialist. In some embodiments, the discharge alert 206 is automatically generated (e.g., automatically generated) in response to receipt of hospital census 202. In some embodiments, the discharge alert 206 is generated and/or transmitted as a fax, an email, a secure email, an attachment to an email, a text message, an instant message, an FTP communication, a webpage for which an address and/or a link is transmitted, a remote portal, a text message, an automated telephone call, a live telephone call, a voicemail, a video communication, a communication over a network, and/or a written communication.

One or more of the subsequent steps of the discharge process 200 may be performed automatically in response to discharge alert 206. For example, one or more of the steps of the discharge process 200 may be performed automatically in response to discharge alert 206.

In one embodiment, the discharge alert 206 is generated at a predetermined frequency, a predetermined day and/or a predetermined time. In one embodiment, the discharge alert 206 is generated daily, such as daily at 9:00 AM. In another embodiment, the discharge alert 206 is generated within a predetermined frequency threshold and/or a predetermined time threshold. In one embodiment, the discharge alert 206 is generated by no later than 9:00 AM daily. In an embodiment, the discharge alert 206 is generated in response to receipt of hospital census 202 and/or upon completion of eligibility check 204.

In some embodiments, such as shown in Step 208, a file for the newly discharged patient identified in the discharge alert 206 is created if the file was not made during the inpatient process 100. In some embodiments, the discharge alert 206 is generated (e.g., automatically generated) if the file was not made during the inpatient process 100. In one embodiment, the file is reviewed to determine if the file was previously made, and the file is generated and/or stored in response to discharge alert 206. The file may be at least partially populated with the data in hospital census 202.

In some embodiments, such as shown in Step 210, mailings to the discharged patient are initiated. As shown in Step 212, a welcome packet explaining the discharge process 200 is sent to a discharged patient identified in Step 206. In one embodiment, the mailings rely on the contact information of the patient obtained in the inpatient process 100. In some embodiments, the welcome packet comprises a hard copy mailing, an email, an email attachment, or a link to a web page, as non-limiting examples. In an embodiment, the welcome packet may be sent to the discharged patient within a time threshold, such as within twenty-four hours of the discharge alert or within forty-eight hours of the discharge alert.

In some embodiments, an initial telephone call 214 with the newly discharged patient is performed. In an embodiment, the initial telephone call 214 is performed within a predetermined time threshold, such as the same day as the discharge alert. Follow-up calls 216 may be conducted at predetermined time periods, such as every three days. In some embodiments, the navigation specialist is prompted to perform initial telephone call 214 upon receipt of a call alert at predetermined time periods, such as daily or every three days, and/or a predetermined time, such as 9:00 AM. In some embodiments, the call alert comprises a fax, an email, a secure email, an attachment to an email, a text message, an instant message, an FTP communication, a webpage for which an address and/or a link is transmitted, a remote portal, a text message, an automated telephone call, a live telephone call, a voicemail, a video communication, a communication over a network, and/or a written communication.

In some embodiments, the call alert is automatically sent at a predetermined time period, such as daily. The call alert may be sent at the predetermined time periods even if no patients are identified in the call alert. The call alert may identify one or more of the patient name, the primary phone number of the patient, date of last contact, name of associated navigation specialist, or the like. The call alert may include a patient that has not been contacted within a number of days that meets a predetermined threshold, such as three days for example, and may include a patient that has been flagged by the system, the navigation specialist, or another user. In some embodiments, the call alert identifies one or more patients that have been unreachable for a number of attempts that meets a predetermined threshold, such as three attempts.

In some embodiments, the navigation specialist confirms that the patient understands the orders for post-discharge care, identifies issues related to the orders for post-discharge care, provides clarification of the orders for post-discharge care, may identify any barriers in facilitating the orders for post-discharge care, and confirms expectations regarding follow-up appointments and the post-discharge timeline. In an embodiment, instructions related to the telephone calls are provided to the navigation specialist. In one embodiment, the instructions are transmitted to the navigation specialist in discharge alert 206 and/or in a message generated in response to discharge alert 206.

In some embodiments, a call 217 prompted by a call alert in Step 216 is postponed and/or rescheduled to another future date. In some embodiments, the call 217 is rescheduled or postponed if the call would have ordinarily been scheduled for a non-working date, such as a weekend or a holiday. In other embodiments, the call 217 is rescheduled or postponed if the call would have ordinarily been scheduled on a date that the patient is known to be unavailable, for example if the patient is already scheduled to have a procedure or other appointment on that date.

In some embodiments, as shown in Step 222, the discharge alert 206 and/or the discharge orders are reviewed. In some embodiments, the discharge alert 208 and/or the discharge orders are reviewed upon their availability. In some embodiments, the discharge alert 206 and/or the discharge orders are electronically displayed to the patient and/or the navigation specialist, for example on a web page In an embodiment, the navigation specialist reviews the discharge summary and/or the discharge orders. In a related embodiment, instructions for reviewing the discharge summary and/or the discharge orders are provided, for example to the navigation specialist. In some embodiments, the instructions are provided in the discharge alert 206.

In some embodiments, as shown in Step 224, the patient chart is created if the patient chart was not created during the inpatient process 100. In some embodiments, the patient chart identifies status changes of the patient, measurements of parameters of the patient, and the like, and may be updated periodically. In an embodiment, the patient chart is created at least in part by the navigation specialist and/or the system. In some embodiments, instructions for creating the patient chart are provided, for example to the navigation specialist. In some embodiments, the instructions are transmitted in the discharge alert 206 and/or in a message generated in response to the discharge alert 206.

In some embodiments, as shown in Step 226, the discharged patient is confirmed to be enrolled in health plan case management. In an embodiment, the discharged patient is confirmed to be enrolled in health plan case management within a predetermined time threshold, such as within forty-eight hours of discharge alert 206. In an embodiment, enrollment in health plan case management is confirmed before discharge alert 206 is generated, such as when the patient is admitted to the hospital, before the procedure, before the discharge, and/or when the patient is discharged, for example. In some embodiments, if the discharged patient is enrolled in health plan case management, the system contacts and interacts with the health plan case management to facilitate meeting the needs of the discharged patient.

In some embodiments, the system and/or the navigation specialist may facilitate orders for post-discharge care, such as by contacting doctors and vendors. Non-limiting examples of vendors include physical therapists, home healthcare entities, durable medical equipment (DME) vendors, and the like. In addition, any medical records that were not available at the time of discharge may be obtained and/or distributed by the system and/or by the navigation specialist. In some embodiments, for example as shown in Step 232, post-discharge medical appointments are scheduled and/or confirmed. In some embodiments, the medical records are distributed as needed to physicians in different disciplines. In some embodiments, the post-discharge medical appointments may be identified on a web page accessible to the patient, the navigation specialist. In an embodiment, the system and/or the navigation specialist may schedule transportation for the post-discharge medical appointments.

In some embodiments, the navigation specialist facilitates a videoconference between the navigation specialist, the patient and a third party, such as a doctor or a vendor. In some embodiments, the videoconference is performed using the system. In some embodiments, the system and/or the navigation specialist contacts the doctors and/or the vendors and/or distributes the records in a fax, an email, a secure email, an attachment to an email, a text message, an instant message, an FTP communication, a webpage for which an address and/or a link is transmitted, a remote portal, a text message, an automated telephone call, a live telephone call, a voicemail, a video communication, a communication over a network, and/or a written communication.

In some embodiments, any or each of Step 228, Step 230 and Step 232 may be performed within a predetermined time threshold, such as within twenty-four hours of the discharge alert of Step 206, within forty-eight hours of the discharge alert of Step 206, or within seventy-two hours after the discharge alert of Step 206.

In some embodiments, a medical appointment alert 234 is generated and/or transmitted to the patient. In some embodiments, the medical appointment alert 234 identifies the date and time of the medical appointment. In some embodiments, the medical appointment alert 234 is sent on the day of or prior to the appointment. In some embodiments, the medical appointment alert 234 is sent even if no appointments are scheduled for that time period, such as for that day. In some embodiments, the medical appointment alert 234 identifies the patient name, the telephone number, the appointment time and the provider. In some embodiments, the medical appointment alert 234 is automatically transmitted to the patient in response to a predetermined time period, such as daily, or after completion of another step, such as Step 232.

In some embodiments, the medical appointment alert 234 is generated by the system and/or provided to the patient. In some embodiments, the medical appointment alert 234 comprises a fax, an email, a secure email, an attachment to an email, a text message, an instant message, an FTP communication, a webpage for which an address and/or a link is transmitted, a remote portal, a text message, an automated telephone call, a live telephone call, a voicemail, a video communication, a communication over a network, and/or a written communication.

In some embodiments, a notification 235 is sent to the patient. In some embodiments, the notification 235 comprises information associated with or from the medical appointment alert 234. In some embodiments, the notification is sent electronically and/or is electronically displayed to the patient, for example on a web site.

In some embodiments, an additional medical appointment is scheduled, for example a follow-up medical appointment scheduled based on the previous medical appointment. In some embodiments, as shown in Step 236, the additional medical appointment is scheduled and/or confirmed. In one embodiment, the additional medical appointment is confirmed before the date on which the additional medical appointment is scheduled. In some embodiments, an alert is sent to the navigation specialist and/or the patient that identifies the date and time of the additional medical appointment. In some embodiments, the additional appointment alert is sent from the system to the patient and/or the navigation specialist identifying the patient name, the telephone number, the appointment time and the provider. In an embodiment, transportation for the additional medical appointment is also coordinated, for example by the navigation specialist.

As shown in Step 237, the navigation specialist or the system may confirm that the appointment was conducted and/or may request the medical records resulting from the appointment after a scheduled appointment with a doctor, a vendor or another healthcare-related individual. In some embodiments, the medical records are distributed to other physicians, such as physicians in different disciplines. In some embodiments, the patient files are updated accordingly. In some embodiments, completion of the appointment is electronically displayed to the navigation specialist and/or the patient, for example on a web site. If the appointment was not completed, the navigation specialist and/or the system may reschedule the appointment and inform the patient of the rescheduled appointment.

In some embodiments, such as shown in Step 238, medical records associated with the additional medical appointment are requested, for example by the system and/or by the navigation specialist. In some embodiments, the medical records are distributed to other physicians as needed, including to physicians in different disciplines. In some embodiments, Step 236 and/or Step 238 comprise transmission of a fax, an email, a secure email, an attachment to an email, a text message, an instant message, an FTP communication, a webpage for which an address and/or a link is transmitted, a remote portal, a text message, an automated telephone call, a live telephone call, a voicemail, a video communication, a communication over a network, and/or a written communication.

In some embodiments, request of the medical records in Step 238 prompts generation of a request alert 239. In some embodiments, the request alert 239 is automatically generated upon request of the medical records in Step 238. In some embodiments, the medical records are distributed in a fax, an email, a secure email, an attachment to an email, a text message, an instant message, an FTP communication, a webpage for which an address and/or a link is transmitted, a remote portal, a text message, an automated telephone call, a live telephone call, a voicemail, a video communication, a communication over a network, and/or a written communication.

In some embodiments, such as shown in Step 241, a record is received from a follow up appointment provider, and the file is updated to include the received record. In some embodiments, the updated file is stored on the system. In some embodiments, the file is updated in Step 241 automatically in response to receipt of the record from the follow up appointment provider. In some embodiments, at least a portion of the updated file is electronically displayed to the patient and/or the navigation specialist, for example on a web site.

In some embodiments, a medication profile 240 for the newly discharged patent is generated. In some embodiments, the navigation specialist confirms that all prescriptions have been filled and obtained by the patient. If any of the prescriptions have not been filled, the navigation specialist may additionally contact the pharmacy or entity that manages the pharmacy benefit plan to ensure that the prescriptions are timely filled. If any of the filled prescriptions have not been obtained by the discharged patient, the navigation specialist may additionally contact the patient to facilitate the patient obtaining the filled prescriptions. In an embodiment, the medication profile is generated within a predetermined time threshold, such as within twenty-four hours of discharge alert 206. In an embodiment, the medication profile 240 is generated at least in part before discharge alert 206, such as before the patient is admitted to the hospital, when the patient is admitted to the hospital, before the procedure, before the discharge, and/or when the patient is discharged, for example, and then may be updated periodically and/or as needed.

In some embodiments, as shown in Step 242, medication profile 240 is reviewed to determine whether reconciliation is needed. For example, a medication regimen prescribed at the time of discharge may inadvertently omit needed medications, unnecessarily duplicate existing therapies, or contain incorrect dosages. In an embodiment, the navigation specialist reviews the medication profile 240 and determines if a medication reconciliation is needed. In some embodiments, the review and determination are performed within a predetermined time threshold, such as within twenty-four hours of discharge alert 206.

If review of the medication profile 240 in Step 242 results in a determination that a medication reconciliation is needed, medication reconciliation 244 is then coordinated, for example by the system and/or by the navigation specialist. In some embodiments, the medication reconciliation 244 comprises a pharmacy consultation. In some embodiments, the navigation specialist may facilitate a videoconference between the navigation specialist, the patient and the pharmacist. In some embodiments, the videoconference is performed using the system. In some embodiments, the pharmacy consultation for the patient is coordinated (e.g., scheduled) using a fax, an email, a secure email, an attachment to an email, a text message, an instant message, an FTP communication, a webpage for which an address and/or a link is transmitted, a remote portal, a text message, an automated telephone call, a live telephone call, a voicemail, a video communication, a communication over a network, and/or a written communication.

In some embodiments, a home visit screen 246 is performed to determine whether the patient requires a home visit. In some embodiments, home visit screen 246 is performed within a predetermined time threshold, such as within forty-eight hours after discharge alert 206. In an embodiment, home visit screen 246 is performed before discharge alert 206, such as when the patient is admitted to the hospital, before the procedure, before the discharge, and/or when the patient is discharged, for example. In some embodiments, a follow-up home visit determination is performed after discharge alert 206 instead or in addition to home visit screen 246.

If home visit screen 246 indicates that the patient requires a home visit, a home visit is scheduled for the patient in Step 248. In some embodiments, the home visit for the patient is scheduled using a fax, an email, a secure email, an attachment to an email, a text message, an instant message, an FTP communication, a webpage for which an address and/or a link is transmitted, a remote portal, a text message, an automated telephone call, a live telephone call, a voicemail, a video communication, a communication over a network, and/or a written communication.

In some embodiments, a post-procedure service comprises one or more of Steps 206-248. For example, one or more of Steps 206-248 may be performed after the patient has been discharged and/or after the system has been notified of completion of the procedure. In some embodiments, a closing alert is generated after a predetermined time period, such as, for example, thirty days after the discharge.

In some embodiments, a patient satisfaction survey 250 is sent to the patient in some embodiments. In an embodiment, patient satisfaction survey 250 is sent in response to discharge alert 206 and/or in response to a closing alert. In some embodiments, patient satisfaction survey 250 comprises a hard copy mailing, an email, an email attachment, or a link to a web page. In an embodiment, patient satisfaction survey 250 is sent within a predetermined time threshold, such as within two weeks of discharge 206.

In some embodiments, a survey alert 260 is generated. In some embodiments, the survey alert 260 is sent to the patient and/or to the navigation specialist to indicate that patient satisfaction survey 250 has been sent. In some embodiments, survey alert 260 is electronically displayed to the patient and/or to the navigation specialist, for example on a web site.

In some embodiments, a final communication 252 is performed with the patient in response to the post discharge call 214. In some embodiments, the final communication 252 is performed at a predetermined time period, such as, for example, thirty days after discharge alert 206. In an embodiment, the final communication 252 is performed in response to or upon receipt of discharge alert 206 and/or in response to or upon receipt of the closing alert. Based at least in part on final communication 252, a final case summary 254 is generated. In some embodiments, the final case summary 254 is input into the system.

In some embodiments, such as shown in Step 256, final case summary 254 is submitted to a supervisor for review and approval. If the supervisor approves the final case summary, the case is closed, as shown in Step 258. In some embodiments, the patient is notified of the case being closed in a communication and/or by electronically displaying a case closed message to the patient, for example on a web site.

In some embodiments, an internal closing process comprises one or more of Step 252, Step 254, Step 256 and Step 258. In some embodiments, final communication 252, final case summary 254 and/or the notification to the patient of the case being closed comprises a fax, an email, a secure email, an attachment to an email, a text message, an instant message, an FTP communication, a webpage for which an address and/or a link is transmitted, a remote portal, a text message, an automated telephone call, a live telephone call, a voicemail, a video communication, a communication over a network, and/or a written communication.

In an embodiment, information related to the healthcare of the patient and progress of the patient is electronically displayed, for example on a web page accessible to the navigation specialist and/or the patient throughout at least a portion of the discharge process 200.

The care assist process 300 may be used for patients that are hospitalized without an overnight stay (outpatient treatment). FIGS. 3A-3B is a flowchart of one embodiment of the care assist process 300. Although the care assist process 300 is described with reference to the flowchart illustrated in FIGS. 3A-3B, other methods of performing the care assist process 300 may be used. For example, unless explicitly stated otherwise, the order of the steps may be changed, and each of the steps is individually optional. One or more of the steps of the care assist process 300 may be performed automatically by the system.

In some embodiments, an outpatient report 302 is generated or obtained, for example from a third party. In one embodiment, the third party is or is controlled by an insurance provider, such as a healthgroup.

In some embodiments, such as shown in Step 304, outpatient report 302 is downloaded, received, obtained or otherwise imported by or into the system. In some embodiments, outpatient report 302 is obtained and/or received automatically at a predetermined time period, such as daily, and optionally at a predetermined time. In some embodiments, outpatient report 302 is obtained as a fax, an email, a secure email, an attachment to an email, a text message, an instant message, an FTP communication, a webpage for which an address and/or a link is transmitted, a remote portal, a text message, an automated telephone call, a live telephone call, a voicemail, a video communication, a communication over a network, and/or a written communication.

In some embodiments, outpatient report 302 comprises a predetermined format. For example, in some embodiments outpatient report 302 comprises a list of patients and data associated with each of the listed patients. In one embodiment, the list of patients in outpatient report 302 comprises patients that previously registered to participate in the care assist process 300. In some embodiments, the list of patients in outpatient report 302 comprises the patients for whom the procedure at the hospital has already been identified. In some embodiments, the list of patients in outpatient report 302 comprises patients receiving outpatient treatment. In some embodiments, eligibility of the listed patients for the subsequent steps of the care assistance process 300 is confirmed before sending outpatient report 302 to the system. In some embodiments, eligibility is based at least in part on the status of the patient, the procedure to be received by the patient, and/or the like.

In some embodiments, as shown in Step 314, outpatient report 302 is processed to identify one or more patients scheduled for procedures for which the care assistance process 300 is appropriate. In some embodiments, outpatient report 302 is processed to confirm that the procedure for the patient is an outpatient procedure and/or a procedure for which the system provides assistance. In some embodiments, outpatient report 302 is automatically obtained and/or automatically processed upon receipt. In one embodiment, eligibility is precluded if the procedure is obstetrical, a lab procedure, an imaging procedure, or a procedure performed at an imaging facility, a sleep center or a doctor's office. In one embodiment, eligibility is based at least in part on one or more of: the procedure being performed in an ambulatory surgery center or hospital outpatient setting, the procedure being authorized by appropriate third party, or the patient being eligible under the patient's health plan.

In some embodiments, such as shown in Step 316, eligibility of the patient(s) identified in outpatient report 302 is confirmed. In some embodiments, eligibility is based at least in part on the insurance coverage of the patient and/or on the type of health insurance owned by the patient, if any. In an embodiment, the system processes (e.g., automatically processes) outpatient report 302 to confirm the eligibility of the patient.

In some embodiments, a patient identification process comprises one or more of Step 304, Step 314, and Step 316. In some embodiments, Step 304, Step 314, and/or Step 316 are performed the same day that the patient is identified in outpatient report 302.

In some embodiments, as shown in Step 318, authorization for the procedure that the patient is scheduled to receive is confirmed. In some embodiments, the file is updated to contain the authorization (or to reflect that authorization was not obtained). In Step 320, the scheduled procedure is confirmed with a health plan physician and a medical facility. In Step 322, the patient is optionally identified as an outside-of-area (OOA) case, for example when the procedure is not covered by the patient's health insurance plan. In Step 324, the patient's enrollment in a health plan case management is confirmed. If the patient is enrolled in health plan case management, the health plan case management may optionally be contacted to facilitate meeting the needs of the patient.

In some embodiments, as shown in Step 326, data associated with the patient is input and stored in a file for the patient, for example in an electronic file stored in the system. In some embodiments, a file (e.g., an electronic file) is generated and stored for each of the patients listed in the outpatient report that are identified in Step 316 as eligible. In some embodiments, the file is populated at least in part with the data from outpatient report 302, and may optionally be populated at least in part with the data obtained when the patient registered for participation in the care assist process 300. In some embodiments, the file is automatically generated and/or stored upon completion of any one of Steps 302-324, for example upon identification of a patient as eligible in Step 316.

In some embodiments, a data input process comprises one or more of Step 318, Step 320, Step 322, Step 324 and Step 326. In some embodiments, Step 318, Step 320, Step 322, Step 324 and/or Step 326 are all performed the same day that the patient is identified in outpatient report 302.

In some embodiments, a new patient alert 328a is generated upon completion of the data input process. In some embodiments, a new case alert 328a is generated for a patient identified as eligible in Step 316 for subsequent steps of the care assistance process 300. In some embodiments, a new patient alert 328a is generated for each patient identified as eligible in Step 316 for subsequent steps of the care assistance process 300. In some embodiments, a new patient alert 328a is generated for a patient whose case has been opened and data input into the system in Step 326. In some embodiments, a new case alert 328a is generated for each patient whose case has been opened and data input into the system in Step 326. In some embodiments, new patient alert 328a is automatically generated by the system, for example upon completion of the data input step (Step 326). In some embodiments, the new patient alert and/or messages in response to new case alert 328a are generated and transmitted to the navigation specialist. In some embodiments, the new patient alert is automatically generated in response to outpatient report 302. In some embodiments, new patient alert 328a and/or messages generated in response to new patient alert 328a are transmitted in a fax, an email, a secure email, an attachment to an email, a text message, an instant message, an FTP communication, a webpage for which an address and/or a link is transmitted, a remote portal, a text message, an automated telephone call, a live telephone call, a voicemail, a video communication, a communication over a network, and/or a written communication.

In some embodiments, a welcome packet 328b explaining the care assistance process 300 is sent to the patient in some embodiments. In some embodiments, welcome packet 328b comprises a hard copy mailing, an email, an email attachment, or a link to a web page. In an embodiment, welcome packet 328b is sent to the patient within a time threshold, such as within twenty-four hours of new patient alert 328a.

In some embodiments, an initial telephone call 332 to the patient is performed, for example by the navigation specialist. In some embodiments, an initial call alert is sent to the navigation specialist to prompt the initial telephone call 332 to the patient. In some embodiments, the initial call alert is automatically generated and/or sent in response to new patient alert 328a. In some embodiments, the initial call alert is generated and/or transmitted as a fax, an email, a secure email, an attachment to an email, a text message, an instant message, an FTP communication, a webpage for which an address and/or a link is transmitted, a remote portal, a text message, an automated telephone call, a live telephone call, a voicemail, a video communication, a communication over a network, and/or a written communication.

In the initial telephone call 332 to the patient, the navigation specialist may confirm: the procedure, including the specific procedure, the location of the procedure, the day and time of the procedure, and/or the individuals involved in providing the procedure. In some embodiments, the initial telephone call may also: provide contact information associated with the navigation specialist; explain the care assist process 300; confirm contact information of the patient, such as telephone number; confirm that the patient understands the procedure that is scheduled; confirm that the patient understands their discharge needs and any post-procedure doctor orders; confirm what prescriptions will be prescribed for administration after the procedure; identify appointments before the procedure, including their time and their location; identify any third party vendors needed after procedure; identify the specialist, if any; identify any ancillary provider; and/or obtain demographics associated with the patient. In some embodiments, the initial telephone call 332 may also confirm the primary care physician, if any, of the patient, as shown in Step 334. In some embodiments, initial telephone call 332 also confirms that the patient has appropriate transportation, including transportation home after discharge, as shown in Step 336. In some embodiments, the file for the patient is updated to include any relevant information (including changes to existing information) obtained during initial telephone call 332.

In some embodiments, initial telephone call 332 is conducted within a predetermined time threshold. In some embodiments, initial telephone call 332 is conducted on the same day as new patient alert 328a for the patient. In some embodiments, initial telephone call 332 is conducted before the procedure. In one embodiment, instructions related to the initial telephone call 332 are provided (e.g., transmitted) to the navigation specialist. In some embodiments, the instructions are transmitted to the navigation specialist in the new patient alert 328a and/or in the call alert.

In some embodiments, information from initial telephone call 332 is entered into the system. Information from initial telephone call 332 may be transmitted to, obtained by and/or stored by the system. In some embodiments, the information is organized and at least a portion thereof is electronically displayed to the navigation specialist and/or the patient for example on a web page.

In some embodiments, one or more follow-up calls 360 may be conducted with the patient. In some embodiments, one or more follow-up calls 360 are conducted at predetermined time periods, such as every three days. In some embodiments, one or more follow-up calls 360 comprise a message, such as an email, a text message, an automated telephone call and/or a live telephone call, requesting that the patient call the navigation specialist if any questions or issues have arisen. In some embodiments, the system is configured to enable a user (e.g., a navigation specialist) to provide assistance (e.g., real-time assistance) to the patient. In some embodiments, a call alert prompts contacting the patient to request that the patient call the system user (e.g., a navigation specialist). In some embodiments, the call alert identifies one or more of the patient name, the primary phone number of the patient, date of last contact, name of associated navigation specialist, or the like. In some embodiments, the call alert is associated with a patient that has not been contacted within a number of days that meets a predetermined threshold, such as three days for example. In some embodiments, the call alert is associated with a patient that has been flagged (e.g., identified) by the system or by the navigation specialist. In some embodiments, the call alert is associated with a patient that has been unreachable for a number of attempts that meets a predetermined threshold, such as three attempts.

In some embodiments, the call alerts are sent at predetermined time periods, such as daily; at predetermined times, such as 9:00 AM, and/or at predetermined patient-related thresholds. In some embodiments, the initial call alert and/or the follow-up call alerts comprise a fax, an email, a secure email, an attachment to an email, a text message, an instant message, an FTP communication, a webpage for which an address and/or a link is transmitted, a remote portal, a text message, an automated telephone call, a live telephone call, a voicemail, a video communication, a communication over a network, and/or a written communication.

In some embodiments, information from the initial telephone call 332 and/or the one or more follow-up calls 360 is entered into the system, for example by updating the file (e.g., the electronic file). In one embodiment, the navigation specialist enters the information. In some embodiments, the information is organized and/or electronically displayed to the navigation specialist and/or to the patient, for example on a web page. In some embodiments, the system is configured to automatically organize the information upon receipt. In some embodiments, the information comprises a fax, an email, a secure email, an attachment to an email, a text message, an instant message, an FTP communication, a webpage for which an address and/or a link is transmitted, a remote portal, a text message, an automated telephone call, a live telephone call, a voicemail, a video communication, a communication over a network, and/or a written communication.

In some embodiments, pre-procedure healthcare and orders 340 are coordinated, for example by a navigation specialist. In some embodiments, pre-procedure healthcare and orders 340 are coordinated using a fax, an email, a secure email, an attachment to an email, a text message, an instant message, an FTP communication, a webpage for which an address and/or a link is transmitted, a remote portal, a text message, an automated telephone call, a live telephone call, a voicemail, a video communication, a communication over a network, and/or a written communication.

In some embodiments, coordinating pre-procedure healthcare and orders 340 additionally includes creating and/or updating a patient chart. In some embodiments, the patient chart is created or updated within a predetermined time threshold, such as within forty-eight hours of initial telephone call 332. The updated patient chart may identify any status changes associated with the patient, measurements of parameters associated with the patient, information related to clinicals, doctor orders, additional information and the like. The patient chart may be updated periodically. In an embodiment, the patient chart is created at least part by the navigation specialist and/or the system.

In one embodiment, instructions for creating the patient chart are transmitted to the navigation specialist. In some embodiments, the instructions are transmitted in new patient alert 332 and/or in a message generated in response to new patient alert 332.

In some embodiments, information from the patient chart, including updates to the patient chart, is transmitted to, obtained by and/or stored by the system. In some embodiments, the system organizes (e.g., automatically organizes) information from the patient chart. In some embodiments, at least a portion of the information from the patient chart is electronically displayed to the navigation specialist and/or to the patient, for example on a web site. In some embodiments, the patient chart is incorporated into the file for the patient stored by the system.

In some embodiments, a daily alert 342 is generated. In some embodiments, daily alert 340 comprises at least a portion of the most recent outpatient report 302. In some embodiments, daily alert 340 identifies at least one of the patient name, the patient phone number, the procedure, the facility, and the provider. In some embodiments, daily alert 340 is automatically sent at a predetermined time period, such as daily, and/or at a predetermined time, such as 9:00 AM. In some embodiments, daily alert 340 is associated with a specific facility and identifies all patients receiving an outpatient procedure the day of or prior to daily alert 340. In an embodiment, daily alert 340 is generated at a predetermined time, for example at 9:00 AM daily. In another embodiment, daily alert 340 is generated within a predetermined time threshold, for example by no later than 9:00 AM daily.

In some embodiments, such as shown in Step 344, daily alert 340 is sent to the navigation specialist and/or to the system. In some embodiments, daily alert 340 is sent as a fax, an email, a secure email, an attachment to an email, a text message, an instant message, an FTP communication, a webpage for which an address and/or a link is transmitted, a remote portal, a text message, an automated telephone call, a live telephone call, a voicemail, a video communication, a communication over a network, and/or a written communication.

In some embodiments, an additional medical appointment 346, for example a follow-up medical appointment, is scheduled based on the previous medical appointment. In some embodiments, the additional medical appointment 346 is scheduled and/or confirmed. In one embodiment, the additional medical appointment 346 is confirmed before the date on which the additional medical appointment 346 is scheduled. In some embodiments, an additional appointment alert 346a is sent to the navigation specialist and/or the patient that identifies the date and time of the additional medical appointment. In some embodiments, the additional appointment alert 346a is sent from the system to the patient and/or the navigation specialist identifying the patient name, the telephone number, the appointment time and the provider. In an embodiment, transportation for the additional medical appointment 346 is also coordinated, for example by the navigation specialist.

After a scheduled appointment with a doctor, a vendor or another healthcare-related individual, confirmation that the appointment was conducted is obtained as shown in Step 351a and 351b. Medical records associated with the scheduled appointment(s) are requested in Steps 352a and 352b, and the obtained records are distributed as needed to other physicians, such as physicians in different disciplines. In some embodiments, medical records necessary for the procedure and/or the post-procedure additional appointments (e.g., such as initial evaluations, pre-procedure doctor orders, and the like) are obtained and/or distributed. Necessary medical records are optionally identified in daily alert 344.

In some embodiments, the navigation specialist assists in facilitating orders for post-procedure care, such as by contacting doctors and vendors. Non-limiting examples of vendors include physical therapists, home healthcare entities, durable medical equipment (DME) vendors, and the like. In some embodiments, the navigation specialist facilitates a videoconference between the navigation specialist, the patient and a third party, such as a doctor or a vendor. In some embodiments, the videoconference is performed using the system.

The patient's file is updated accordingly, as shown in Steps 354a and 354b. In some embodiments, completion of the appointment is electronically displayed to the navigation specialist and/or the patient, for example on a web page. If the appointment was not completed, the appointment may optionally be rescheduled and, if necessary, the patient is notified of the rescheduled appointment.

In some embodiments, a request alert 353 is generated requesting a medical record associated with the procedure. In some embodiments, the request alert 353 is generated on the same date or on a later date than the date on which the procedure is scheduled. In some embodiments, the request alert 353 is automatically generated upon completion of one or more previous steps in care assist process 300, for example after confirmation that the patient attended the procedure appointment is received in Step 351a. In some embodiments, request alert 353 is in an electronic format and is transmitted electronically to the follow up appointment provider. In some embodiments, request alert 353 comprises a fax, an email, a secure email, an attachment to an email, a text message, an instant message, an FTP communication, a webpage for which an address and/or a link is transmitted, a remote portal, a text message, an automated telephone call, a live telephone call, a voicemail, a video communication, a communication over a network, and/or a written communication.

As shown in Steps 354a and 354b, medical records resulting from the procedure and/or the additional procedure are obtained and/or distributed to the primary care physician, another entity associated with a follow-up appointment after the procedure, and the like. If the patient does not have a primary care physician, the navigation specialist may coordinate a primary care physician for the patient as part of Steps 354a and/or 354b. In some embodiments, the navigation specialist facilitates a videoconference between the navigation specialist, the patient and a primary care physician participate. In some embodiments, the videoconference is performed using the system. In some embodiments, the medical records are distributed as needed to other physicians, such as physicians in different disciplines.

In some embodiments, Step 348, Step 350, Step 352 and/or Step 354 comprise transmitting information using a fax, an email, a secure email, an attachment to an email, a text message, an instant message, an FTP communication, a webpage for which an address and/or a link is transmitted, a remote portal, a text message, an automated telephone call, a live telephone call, a voicemail, a video communication, a communication over a network, and/or a written communication.

In some embodiments, a weekly alert 362 is generated and/or transmitted to the navigation specialist. In some embodiments, weekly alert 362 comprises at least a portion of the most recent outpatient report 302. In some embodiments, weekly alert 362 identifies at least one of the patient name, the patient phone number, the procedure, the facility, the provider or the like. In some embodiments, weekly alert 362 identifies all patients receiving an outpatient procedure at a facility for the current week, excluding the current day and prior days. In some embodiments, weekly alert 362 is sent at a predetermined time period, such as daily, and/or at a predetermined time, such as 9:00 AM. In some embodiments, weekly alert 362 comprises a fax, an email, a secure email, an attachment to an email, a text message, an instant message, an FTP communication, a webpage for which an address and/or a link is transmitted, a remote portal, a text message, an automated telephone call, a live telephone call, a voicemail, a video communication, a communication over a network, and/or a written communication.

In response to weekly alert 362, services for patients who have not received the procedure yet are coordinated. In some embodiments, as shown in Step 364, the pre-procedure medical appointments and initial evaluations are confirmed. In some embodiments, a pre-procedure medical appointment alert 365 that identifies the date and time of the pre-procedure medical appointment is sent to the patient. In some embodiments, the medical appointment alert is sent on the day of or prior to the pre-procedure medical appointment. In some embodiments, the pre-procedure medical appointment alert is sent to the patient in Step 367, for example in an email or a text message, or by performing an automated telephone call or a live telephone call. In some embodiments, the pre-procedure medical appointment alert is electronically displayed to the navigation specialist and/or to the patient, for example on a web page.

In some embodiments, such as shown in Step 366, medical records necessary for the pre-procedure medical appointment and any other information needed for the pre-procedure medical appointment are provided, for example by the navigation specialist.

In some embodiments, Step 364 and/or Step 366 use a fax, an email, a secure email, an attachment to an email, a text message, an instant message, an FTP communication, a webpage for which an address and/or a link is transmitted, a remote portal, a text message, an automated telephone call, a live telephone call, a voicemail, a video communication, a communication over a network, and/or a written communication.

In some embodiments, a future alert 356 is generated and/or provided. In some embodiments, future alert 356 comprises at least a portion of the information from the most recent outpatient report 302. In some embodiments, future alert 356 identifies at least one of the patient name, the patient phone number, the procedure, the facility, the provider or the like. In one embodiment, future alert 356 identifies all patients receiving an outpatient procedure at a facility for the week directly following the current week. In some embodiments, future alert 356 is sent at a predetermined time period, such as daily, and/or at a predetermined time, such as 9:00 AM. In some embodiments, future alert 356 comprises a fax, an email, a secure email, an attachment to an email, a text message, an instant message, an FTP communication, a webpage for which an address and/or a link is transmitted, a remote portal, a text message, an automated telephone call, a live telephone call, a voicemail, a video communication, a communication over a network, and/or a written communication.

In some embodiments, services for patients who have not been admitted yet and/or have not received the procedure yet are coordinated, for example by a navigation specialist. For example, in Step 358, prescriptions for the patient are confirmed to be available to fill prior to the procedure. In some embodiments, the navigation specialist facilitates a videoconference between the navigation specialist, the patient and the pharmacist. In some embodiments, the videoconference is performed using the system.

In various embodiments, prescriptions for patients who have not been admitted yet and/or have not received the procedure yet are coordinated in Step 358 using a fax, an email, a secure email, an attachment to an email, a text message, an instant message, an FTP communication, a webpage for which an address and/or a link is transmitted, a remote portal, a text message, an automated telephone call, a live telephone call, a voicemail, a video communication, a communication over a network, and/or a written communication.

In various embodiments, at least one of daily alert 340, weekly alert 362 and/or the future alert 356 are generated (e.g., automatically generated) in response to predetermined time periods.

In some embodiments, a medication profile is generated for a newly discharged patient. For example, the navigation specialist confirms that all prescriptions have been filled and obtained by the patient. If any of the prescriptions have not been filled, the navigation specialist contacts the pharmacy to ensure that the prescriptions are timely filled. If any of the filled prescriptions have not been obtained by the discharged patient, the navigation specialist contacts the patient to facilitate the patient obtaining the filled prescriptions. In an embodiment, the medication profile is generated within a predetermined time threshold, such as within forty-eight hours of the procedure.

In some embodiments, the navigation specialist identifies whether a medication reconciliation is needed. For example, a medication regimen prescribed at the time of discharge may inadvertently omit needed medications, unnecessarily duplicate existing therapies, or contain incorrect dosages. In an embodiment, the need for a medication reconciliation is determined within a predetermined time threshold, such as within forty-eight hours of the procedure.

If a medication reconciliation is needed, the need for a pharmacy consultation for the discharged patient is determined. In some embodiments, the pharmacy consultation is scheduled for the patient, for example by the navigation specialist. The navigation specialist may facilitate a videoconference between the navigation specialist, the patient and the pharmacist. In some embodiments, the videoconference is performed using the system. In some embodiments, the pharmacy consultation for the patient is scheduled using a fax, an email, a secure email, an attachment to an email, a text message, an instant message, an FTP communication, a webpage for which an address and/or a link is transmitted, a remote portal, a text message, an automated telephone call, a live telephone call, a voicemail, a video communication, a communication over a network, and/or a written communication.

In some embodiments, a final communication 372 with the patient is performed at a predetermined time period, such as, for example, thirty days after the procedure. A final case summary 378 is generated based at least in part on the final communication with the patient. In some embodiments, final case summary 378 is input and/or transmitted to the system.

In some embodiments, a patient satisfaction survey is sent to the patient. In some embodiments, the patient satisfaction survey comprises a hard copy mailing, an email, an email attachment, and/or a link to a web page. In one embodiment, the patient satisfaction survey is sent within a predetermined time threshold, such as within two weeks, of the discharge alert.

In some embodiments, such as shown in Step 380, the final case summary 378 may be submitted to a supervisor for review and approval. If the supervisor approves the final case summary, the case is closed. In some embodiments, the patient is notified of the case being closed. In some embodiments, the final case summary and/or the notification that case is closed comprises a fax, an email, a secure email, an attachment to an email, a text message, an instant message, an FTP communication, a webpage for which an address and/or a link is transmitted, a remote portal, a text message, an automated telephone call, a live telephone call, a voicemail, a video communication, a communication over a network, and/or a written communication.

In some embodiments, an internal closing process comprises one or more of Step 372, 378 and/or 380 may form an internal closing process. In some embodiments, at least a portion of the care assistance process 300 is performed before the procedure. Preferably, the initial telephone call, updating the file to include the results of the initial telephone call, creation of the patient chart, updating the patient chart, coordinating prescriptions to be filled and obtained by the patient, confirmation of pre-procedure appointments and post-procedure appointments, ensuring that pre-procedure orders and test results are received by the appropriate entity, ensuring that the applicable initial evaluations and physical therapies are scheduled, and obtaining the post-procedure orders related to doctors and vendors and distributing the orders appropriately are performed before the procedure. Preferably, follow-up telephone calls, confirming post-procedure appointments with a provider, updating medical records, facilitating post-procedure orders, distributing medical records to follow-up doctors and vendors, generating the medication profile, and identifying cases that require medication reconciliation are performed after the procedure.

In an embodiment, information related to the healthcare of the patient and progress of the patient is electronically displayed to the navigation specialist and/or to the patient partially throughout or throughout the care assistance process 300.

In any embodiment described herein, the term “electronically displayed” includes any display or presentation of information on an electronic screen including, but not limited to, a computer screen, a monitor, a laptop screen, a television, a cellular device screen, a movie screen, a mobile device screen, a tablet screen, and the like.

Any step of any method disclosed herein may be performed automatically upon completion of any prior step, e.g., without requiring instructions or approval from a user (e.g., a navigation specialist).

In one embodiment, the present disclosure provides a method of coordinating a service to a patient from a service provider, the method comprising receiving, in a control device, a first communication comprising information from a hospital, the first communication selected from the group consisting of an electronic hospital census and an outpatient authorization report, wherein the first communication identifies a patient in need of receiving a service; determining, in the control device, the service needed by the patient as a function of at least the first communication, wherein the service is to be provided before a procedure, after a procedure, before a discharge, after a discharge, and combinations thereof; storing the determined service in the control device as a file; and generating, in a second communication, an alert that prompts a navigation specialist to coordinate the determined service for the patient, wherein the patient is selected from the group consisting of an individual admitted to a hospital, an individual scheduled for the service, and combinations thereof, and wherein the second communication is generated at a time selected from the group consisting of before admission to the hospital, at admission to the hospital, before the service, after the service, at discharge, and combinations thereof. In some embodiments, the method further comprises electronically displaying information related to the service to the patient, wherein the information is provided by the control device. In some embodiments, the method further comprises generating a census alert in response to the first communication. In some embodiments, the service comprises prescribing the patient a medication. In some embodiments, the method further comprises verifying that the patient filled the prescription. In some embodiments, the service is a medical appointment. In some embodiments, the method further comprises verifying that the patient attended the medical appointment. In some embodiments, the method further comprises transmitting an alert to the patient prior to the medical appointment, wherein the alert comprises information from the second communication associated with the medical appointment, and wherein the alert is transmitted by an entity that operates the control device. In some embodiments, the service comprises ordering an appointment with and/or a delivery from a vendor. In some embodiments, the method further comprises verifying that the patient attended the appointment and/or received the delivery. In some embodiments, the method further comprises automatically generating a discharge alert when the control device receives information from the first communication identifying that the patient is discharged. In some embodiments, the method further comprises updating the file in response to information input by a navigation specialist. In some embodiments, the method further comprises electronically displaying information regarding a plurality of services comprising the service and an associated completion status for each of the plurality of services, wherein the information regarding the plurality of services is provided by the control device. In some embodiments, the information is electronically displayed to the patient. In some embodiments, the information comprises an aggregate of information associated with a plurality of patients, and wherein the information is electronically displayed to a third party. In some embodiments, the information comprises an aggregate of information associated with a plurality of patients, and wherein the information is electronically displayed to the navigation specialist. In some embodiments, the method further comprises electronically displaying information from the file to the patient. In some embodiments, the method further comprises generating, by the control device, a call alert that requests contact between the patient and a navigation specialist. In some embodiments, the method further comprises generating, by the control device, a no medical appointment alert. In some embodiments, the method further comprises generating, by the control device, a no vendor appointment and/or a no vendor delivery alert. In some embodiments, the method further comprises transmitting a medical record associated with the patient from the control device to a third party associated with the service and tracking the transmission in the control device. In some embodiments, the control device is remotely located relative to the hospital. In some embodiments, the method further comprises transmitting a medical record associated with the patient to a third party associated with another service needed by the patient, wherein the medical record results from the service coordinated by the second communication.

In one embodiment, the present disclosure provides a system configured to coordinate medical services for a patient, the system comprising a computer-readable medium and a display device, wherein the computer-readable medium comprises instructions for receiving, in a control device, a first communication comprising information from a hospital, the first communication selected from the group consisting of an electronic hospital census and an outpatient authorization report, wherein the first communication identifies a patient in need of receiving a service; determining, in the control device, the service needed by the patient as a function of at least the first communication, wherein the service is to be provided before a procedure, after a procedure, before a discharge, after a discharge, and combinations thereof; storing the determined service in the control device as a file; and generating, in a second communication, an alert that prompts a navigation specialist to coordinate the determined service for the patient, wherein the patient is selected from the group consisting of an individual admitted to a hospital, an individual scheduled for the service, and combinations thereof, and wherein the second communication is generated at a time selected from the group consisting of before admission to the hospital, at admission to the hospital, before the service, after the service, at discharge, and combinations thereof.

In some embodiments, the present disclosure provides a non-transitory computer-readable medium that stores instructions that when executed cause a processor to perform at least one step of a method disclosed herein.

In some embodiments, the present disclosure provides a system comprising a control device configured to receive a first communication selected from the group consisting of an electronic hospital census and an outpatient authorization report, wherein the first communication identifies a patient selected from the group consisting of an individual admitted to a hospital, an individual scheduled for an elective outpatient procedure, and combinations thereof, and the control determines a service needed by the patient identified by the first communication, wherein the service is selected from the group consisting of a pre-procedure service, a post-procedure service, a pre-discharge service, a post-discharge service and combinations thereof, wherein the control device stores a file associated with the patient that identifies the service, and wherein the control device is operated by an entity that is a different entity than the hospital; and a navigation specialist communicatively connected to the control device and operated by the entity that operates the control device, wherein at least one of the control device and the navigation specialist is configured to transmit a second communication that coordinates the service from a provider that is a different entity than the entity that operates the control device and the navigation specialist, and wherein the second communication is performed at a time selected from the group consisting of before admission to the hospital, at admission to the hospital, before the service, after the service, at discharge, and combinations thereof. In some embodiments, the navigation specialist is remotely located relative to the control device.

### EXAMPLES

In one embodiment, a method of the present disclosure comprises a plurality of alerts configured to be performed automatically or manually as shown in Table 1, below.

TABLE 1

| | | Manual/ | | |
|---|---|---|---|---|
| | Frequency | Automatic | Excludes | Includes |
| CAP & TCP | Daily, | Automatic | Patients with no call date | Patient with a call type |
| Call Alert | excluding | | | ‘postponed’ with |
| | weekends at | | | corresponding call date equal |
| | 9 am for CAP | | | to date of the alert |
| | and 12 pm for | | Patients with a closed date | All call types |
| | TCP | | Call Reasons without the word | Call Reasons with the word |
| | | | “patient” | “patient” |
| | | | | Call Reasons with the word |
| | | | | “patient” that are aging 3 |
| | | | | business days or more from |
| | | | | date of last call. |
| | | | | All cases with no closed date |
| Heads in | Daily, | Manual | Patients with a discharge date | patients with an admission |
| Beds | exlcuding | | | date not greater than today's |
| | weekends | | | date |
| Future Heads | Daily, | Manual | Patients with a discharge date | patients with an admission |
| in Beds | exlcuding | | | date greater that today's date |
| | weekends | | | |
| CAP New | Daily, | Manual | Patients with a closed date | patients with a notification |
| Patient Alert | exlcuding | | | date equal to current date |
| | weekends | | | |
| CAP Daily | Daily, | Automatic | Patients with a closed date | patients with a procedure date |
| Alert | exlcuding | | | equal to current date |
| | weekends, at | | | |
| | 9 am | | | |
| CAP Weekly | Daily, | Automatic | Patients with a closed date | patients with a procedure date |
| Alert | exlcuding | | | for remaining days of the |
| | weekends and | | | current week (including |
| | Friday, at | | | weekend) greater but not |
| | 11 am | | | equal to current date |
| CAP Future | Daily, | Automatic | Patients with a closed date | patients with a procedure date |
| Alert | excluding | | | that falls between Monday |
| | weekends, at | | | thru Sunday of immediate |
| | 11 am | | | Following week. |
| TCP Survey | Daily, | Automatic | Patients with a survey received | patients with a discharge date |
| Alert | excluding | | date | equal to or greater than 10 |
| | weekends, at | | | business days prior to current |
| | 8 am | | | date |
| | | | | patients with a survey sent |
| | | | | date equal to or greater than |
| | | | | 10 business days prior to |
| | | | | current date |
| Discharge | Daily, | Manual | Patients with a closed date | patients with a discharge date |
| Alert | excluding | | | one day less than or equal to |
| | weekends | | | current date |
| Weekend | Monday | Manual | Patients with a closed date | patients with a discharge date |
| Discharge | | | | equal to current calendar date |
| Alert | | | | and each previous date up to |
| | | | | 3 calendar days prior to |
| | | | | current date |
| Medical | Daily, | Automatic | patients with a medical records | patient with a medical records |
| Records | excluding | | received date; patients with a | request date that is greater |
| Request Alert | weekends, at | | closed date | than 5 days. |
| | 8 am | | Patients with a closed date | patients with a Specialist/PCP |
| CAP & TCP | Daily, | Automatic | | appointment date equal to |
| Follow up | excluding | | | current date + one day. |
| Appointment | weekends, at | | | Monday through Thursday |
| Alerts | 12 pm | | patients with an appointment | patients with a Specialist/PCP |
| | | | status populated | appointment date equal to |
| | | | | todays date + 1, 2 and 3 days |
| | | | | (Saturday, Sunday or |
| | | | | Monday) |
| TCP & CAP | Daily, | Automatic | Appointments with with no | patients with a discharge date |
| No Follow up | excluding | | appointment necessary box | or a procedure date and no |
| Appointment | weekends, at | | marked | Specialist/PCP appointment |
| Alerts | 8 am | | | date |
| | | | Patients with a closed date | |
| CAP Survey | Daily, | Automatic | Patients with a survey received | patients with a procedure date |
| Alert | excluding | | date | equal to or greater than 10 |
| | weekends, at | | | business days prior to current |
| | 8 am | | | date |
| | | | | patients with a survey sent |
| | | | | date equal to or greater than |
| | | | | 10 business days prior to |
| | | | | current date |
| Ancillary | Daily, | Automatic | Patients with a closed date | patients with an initial service |
| Follow up | excluding | | | date equal to current date + |
| Appointment | weekends, at | | | one day. Monday through |
| Alert | 12 pm | | | Thursday |
| | | | | patients with an initial service |
| | | | | date equal to todays date + 1, |
| | | | | 2 and 3 days (Saturday, |
| | | | | Sunday or Monday) |

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A method of coordinating a service to a patient from a service provider, the method comprising:
receiving, in a control device, a first communication comprising information from a hospital, the first communication selected from the group consisting of an electronic hospital census and an outpatient authorization report, wherein the first communication identifies a patient in need of receiving a service;
determining, in the control device, the service needed by the patient as a function of at least the first communication, wherein the service is to be provided before a procedure, after a procedure, before a discharge, after a discharge, and combinations thereof;
storing the determined service in the control device as a file; and
generating, in a second communication, an alert that prompts a navigation specialist to coordinate the determined service for the patient,
wherein the patient is selected from the group consisting of an individual admitted to a hospital, an individual scheduled for the service, and combinations thereof, and
wherein the second communication is generated at a time selected from the group consisting of before admission to the hospital, at admission to the hospital, before the service, after the service, at discharge, and combinations thereof.

2. The method of claim 1, further comprising:
electronically displaying information related to the service to the patient, wherein the information is provided by the control device.

3. The method of claim 1, further comprising:
generating a census alert in response to the first communication.

4. The method of claim 1, wherein the service comprises prescribing the patient a medication.

5. The method of claim 4 further comprising:
verifying that the patient filled the prescription.

6. The method of claim 1, wherein the service is a medical appointment.

7. The method of claim 6 further comprising
verifying that the patient attended the medical appointment.

8. The method of claim 6, further comprising:
transmitting an alert to the patient prior to the medical appointment,
wherein the alert comprises information from the second communication associated with the medical appointment, and
wherein the alert is transmitted by an entity that operates the control device.

9. The method of claim 1, wherein the service comprises ordering an appointment with and/or a delivery from a vendor.

10. The method of claim 9 further comprising:
verifying that the patient attended the appointment and/or received the delivery.

11. The method of claim 1, further comprising:
automatically generating a discharge alert when the control device receives information from the first communication identifying that the patient is discharged.

12. The method of claim 1, further comprising:
updating the file in response to information input by a navigation specialist.

13. The method of claim 1, further comprising:
electronically displaying information regarding a plurality of services comprising the service and an associated completion status for each of the plurality of services, wherein the information regarding the plurality of services is provided by the control device.

14. The method of claim 13, wherein the information is electronically displayed to the patient.

15. The method of claim 13, wherein the information comprises an aggregate of information associated with a plurality of patients, and wherein the information is electronically displayed to a third party.

16. The method of claim 13, wherein the information comprises an aggregate of information associated with a plurality of patients, and wherein the information is electronically displayed to the navigation specialist.

17. The method of claim 1 further comprising:
electronically displaying information from the file to the patient.

18. The method of claim 1 further comprising:
generating, by the control device, a call alert that requests contact between the patient and a navigation specialist.

19. The method of claim 1 further comprising:
generating, by the control device, a no medical appointment alert.

20. The method of claim 1 further comprising:
generating, by the control device, a no vendor appointment and/or a no vendor delivery alert.

21. The method of claim 1 further comprising:
transmitting a medical record associated with the patient from the control device to a third party associated with the service; and
tracking the transmission in the control device.

22. The method of claim 1, wherein the control device is remotely located relative to the hospital.

23. The method of claim 1 further comprising:
transmitting a medical record associated with the patient to a third party associated with another service needed by the patient,
wherein the medical record results from the service coordinated by the second communication.

24. A non-transitory computer-readable medium that stores instructions that when executed cause a processor to perform at least one step of the method of claim 1.

25. A system comprising:
a control device configured to receive a first communication selected from the group consisting of an electronic hospital census and an outpatient authorization report, wherein the first communication identifies a patient selected from the group consisting of an individual admitted to a hospital, an individual scheduled for an elective outpatient procedure, and combinations thereof, and the control determines a service needed by the patient identified by the first communication, wherein the service is selected from the group consisting of a pre-procedure service, a post-procedure service, a pre-discharge service, a post-discharge service and combinations thereof, wherein the control device stores a file associated with the patient that identifies the service, and wherein the control device is operated by an entity that is a different entity than the hospital; and
a navigation specialist communicatively connected to the control device and operated by the entity that operates the control device, wherein at least one of the control device and the navigation specialist is configured to transmit a second communication that coordinates the service from a provider that is a different entity than the entity that operates the control device and the navigation specialist, and wherein the second communication is performed at a time selected from the group consisting of before admission to the hospital, at admission to the hospital, before the service, after the service, at discharge, and combinations thereof.

26. The system of claim 25, wherein the navigation specialist is remotely located relative to the control device.
